(19) United States
(12) Patent Application Publication
(10) Pub. No.: US 2025/0372268 A1
(43) Pub. Date: Dec. 4, 2025

(54) Computational Systems and Methods for Evaluating Eye Health from Behavioral Data

(71) Applicant: Johnson & Johnson Vision Care, Inc., Jacksonville, FL (US)

(72) Inventor: Samuele VISENTINI, Jacksonville, FL (US)

(21) Appl. No.: 19/224,120

(22) Filed: May 30, 2025

Related U.S. Application Data
(60) Provisional application No. 63/654,304, filed on May 31, 2024.

(51) Int. Cl. G16H 50/30; G06V 40/20; G16H 10/60

(52) U.S. Cl. CPC G16H 50/30; G06V 40/20; G16H 10/60

(57) ABSTRACT

Behavioral data regarding a user's use of the device or interaction with the user's environment can be assessed by one or more machine learning models having been trained to segment a plurality of users into patient subgroups differentiated with regard to one or more eye health conditions. The user may be classified into a patient subgroup, and the user may be presented with a notification and/or educational content related to an associated risk. A communicative connection between the user and a system of an eye health care provider can be established, and the user can be provided with an interface for scheduling an in-person appointment with the eye health care provider.

### CROSS-REFERENCE TO RELATED APPLICATIONS

This application claims the benefit of U.S. Provisional Application 63/654,304 filed on May 31, 2024 titled “Machine Learning Systems and Methods for Evaluating Eye Health from Behavioral Data.” The content of this application is incorporated by reference herein.

### BACKGROUND

This disclosure relates generally to technology for making computerized assessments regarding patient eye health.

### SUMMARY

Assessment of a patient's eye health is typically done in the office of an eye care professional, such an optician or ophthalmologist. However, an eye care professional may have limited visibility into the patient's behaviors, which can delay or prevent accurate diagnosis and/or medical device. The patient themself may be unaware of or unable to track such behaviors. Computerized analytic tools (including artificial intelligence (AI)) have the potential to perform assessments of a patient's behavior over time, and use data from those assessments to connect the patient to a health care professional and/or educate and enable the patient to seek healthcare options. Methods and systems directed to analytics to assess patient behavior (including without limitation, their use of an electronic device) are set forth in the accompanying drawings and description below.

In some implementations of the present disclosure, computerized deep-learning systems and methods are disclosed for capturing, via a device, behavioral data regarding (a) a user's use of the device, or (b) the user's interaction with the user's environment; capturing, via the device, input data comprising one or more of: (a) user survey data, (b) user biometric data, (c) data entered by the user through a screen or peripheral of the device, and (d) device sensor data; obtaining, from a remote system, historical data related to one or more of: (a) medical history of the user, (b) medical data relating to a plurality of patients, (c) clinical trial data, and (d) purchase data; providing the historical data to a first machine learning model, the first machine learning model having been trained to segment a plurality of users into patient subgroups that are clinically distinct and associated with meaningful differences with regard to one or more eye health conditions; providing at least the behavioral data and the input data to a second machine learning model, the second machine learning model having been trained to classify the user into one or more of the patient subgroups; and performing an action in response to the classification of the user into the one or more of the patient subgroups, wherein the action comprises one or more of the following: (i) displaying a notification to the user, via a display of the device, of at least one risk associated with the one or more patient subgroups, (ii) presenting to the user, via a display of the device, of educational content associated with the at least one risk, (iii) presenting to the user, via a display of the device, of contact information for a healthcare provider specialized for the one or more eye health conditions, and (iv) establishing a communicative connection between the device and a remote server.

In some implementations of the present disclosure, the behavioral data regarding the user's use of the device may comprise a measurement of time spent viewing the device. In some embodiments, the behavioral data regarding the user's interaction with the user's environment may comprise a measurement of time spent in an outdoors space.

In some implementations of the present disclosure, the remote server may be the computing system or telecommunications system of an eye health care provider. In some embodiments, the remote server may be a purchasing system or a distribution system. In some embodiments, the remote server may be the computing system of an educational provider.

In some implementations of the present disclosure, the performing an action in response to the classification of the user into the one or more of the patient subgroups comprises presenting the user with an interface for scheduling an in-person appointment with the eye health care provider.

In some implementations of the present disclosure, a method may comprise capturing, by a device, behavioral data regarding (a) a user's use of the device, or (b) the user's interaction with the user's environment; capturing, by the device, input data comprising one or more of: (a) user survey data, (b) user biometric data, (c) data entered by the user through a screen or peripheral of the device, and (d) device sensor data; obtaining, from a remote system, historical data related to one or more of: (a) medical history of the user, (b) medical data relating to a plurality of patients, (c) clinical trial data, and (d) purchase data; providing the historical data, the behavioral data, and the input data to a machine learning model, the machine learning model having been trained to generate a health score associated with the user; and displaying a notification to the user, via a display of the device, of information associated with the generated health score.

In some implementations of the present disclosure, the generated health score is any of: an eye health score, an overall health score, a behavioral health score, and a cardiovascular health score.

In some implementations of the present disclosure, a method may comprise capturing, via a device, behavioral data regarding (a) a user's use of the device, or (b) the user's interaction with the user's environment; obtaining, from a first remote system, medical data for the user relating to one or more eye health conditions, wherein the first remote system contains records from at least one eye health care provider; obtaining, from a second remote system, historical data related to one or more of: (a) medical history of the user, (b) medical data relating to a plurality of patients, (c) clinical trial data, and (d) purchase data; providing the historical data, behavioral data, and medical data to a machine learning model, the machine learning model having been trained to generate an eye health score associated with the user; and displaying a notification to the user, via a display of the device, of information associated with the generated eye health score.

In some implementations of the present disclosure, the eye health score represents a progression of an eye condition over time. In some embodiments, the eye health score represents a comparison of an eye health of the user against an eye health value representative of an aggregate patient population.

In some implementations of the present disclosure, a method may comprise capturing, by a device, behavioral data regarding (a) a user's use of the device, or (b) the user's interaction with the user's environment; capturing, by the device, one or more of (i) input data comprising one or more of: (a) user survey data, (b) user biometric data, (c) data entered by the user through a screen or peripheral of the device, and (d) device sensor data and (ii) medical data for the user relating to one or more eye health conditions, wherein the first remote system contains records from at least one eye health care provider; obtaining, from a remote system, historical data related to one or more of: (a) medical history of the user, (b) medical data relating to a plurality of patients, (c) clinical trial data, and (d) purchase data; providing the historical data, the behavioral data, and one or more of the input data and the medical data to a machine learning model, the machine learning model having been trained to generate a health score associated with the user; and displaying a notification to the user, via a display of the device, of information associated with the generated health score.

In some implementations of the present disclosure, a method may comprise capturing, by a device, behavioral data regarding (a) a user's use of the device, or (b) the user's interaction with the user's environment; capturing, by the device, input data comprising one or more of: (a) user survey data, (b) user biometric data, (c) data entered by the user through a screen or peripheral of the device, and (d) device sensor data; applying one or more algorithms to classify the user into one or more patient subgroups that are clinically distinct and associated with meaningful differences with regard to one or more eye health conditions; and performing an action in response to the classification of the user into the one or more of the patient subgroups, wherein the action comprises one or more of the following: (i) displaying a notification to the user, via a display of the device, of at least one risk associated with the one or more patient subgroups, (ii) presenting to the user, via a display of the device, of educational content associated with the at least one risk, (iii) presenting to the user, via a display of the device, of contact information for a healthcare provider specialized for the one or more eye health conditions, and (iv) establishing a communicative connection between the device and a remote server.

In some implementations of the present disclosure, a method may comprise capturing, via a device, behavioral data regarding (a) a user's use of the device, or (b) the user's interaction with the user's environment; providing the behavioral data to a machine learning model, the machine learning model having been trained to classify the user into one or more of the patient subgroups, each patient subgroup being associated with a distinct eye health condition; establishing, in response to the classification of the user into the one or more of the patient subgroups, a communicative connection between the device and a computing system or telecommunications system of an eye health care provider; and providing to the user, in response to the classification of the user into the one or more of the patient subgroups, an interface with an appointment scheduling system for the eye health care provider. In some implementations of the present disclosure, the method may further comprise providing, to the user, a listing of suggested eye health care providers. In some implementations of the present disclosure, the appointment scheduling system is a system for scheduling an in-office appointment with the eye health care provider.

### BRIEF DESCRIPTION OF THE DRAWINGS

The disclosed embodiments have advantages and features that will be more readily apparent from the detailed description, the appended claims, and the accompanying figures (or drawings). The disclosure can be better understood with reference to the following drawings. The elements of the drawings are not necessarily to scale relative to each other, emphasis instead being placed upon clearly illustrating the principles of the disclosure.

FIG. 1A illustrates components of a computerized pipeline system for making eye health assessments, in accordance with some embodiments of the present disclosure.

FIG. 1B illustrates components of a computerized pipeline system for making eye health assessments, in accordance with some embodiments of the present disclosure.

FIG. 1C illustrates components of a computerized pipeline system for making eye health assessments, in accordance with some embodiments of the present disclosure.

FIG. 2 illustrates an example of a computer system one or more of which may be used to implement one or more of the apparatuses, systems, and methods illustrated herein.

FIG. 3 illustrates a flow chart implementation of one or more of the apparatuses, systems, and methods illustrated herein.

FIG. 4A illustrates components of a computerized pipeline system for making eye health assessments, in accordance with some embodiments of the present disclosure.

FIG. 4B illustrates components of a computerized pipeline system for making eye health assessments, in accordance with some embodiments of the present disclosure.

FIGS. 5A, 5B, 5C, and 5D illustrates exemplary user interfaces displayed to a user as part of a system for making eye health assessments, in accordance with some embodiments of the present disclosure.

FIGS. 6A, 6B, 6C, and 6D illustrate exemplary user interfaces displayed to a user as part of a system for making eye health assessments, in accordance with some embodiments of the present disclosure.

FIGS. 7A and 7B illustrate exemplary user interfaces displayed to a user as part of a system for making eye health assessments, in accordance with some embodiments of the present disclosure.

FIGS. 8A, 8B, and 8C illustrate exemplary user interfaces displayed to a user as part of a system for making eye health assessments, in accordance with some embodiments of the present disclosure.

FIGS. 9A, 9B, 9C, and 9D illustrate exemplary user interfaces displayed to a user as part of a system for making eye health assessments, in accordance with some embodiments of the present disclosure.

FIGS. 10A, 10B, 10C, and 10D illustrate exemplary user interfaces displayed to a user as part of a system for making eye health assessments, in accordance with some embodiments of the present disclosure.

FIGS. 11A, 11B, 11C, and 11D illustrate exemplary user interfaces displayed to a user as part of a system for making eye health assessments, in accordance with some embodiments of the present disclosure.

FIG. 12 illustrates components of a network comprising an eye health assessment system, in accordance with some embodiments of the present disclosure.

FIGS. 13A, 13B, and 13C illustrate exemplary user interfaces displayed to a user as part of a computerized pipeline system for making eye health assessments, in accordance with some embodiments of the present disclosure.

FIGS. 14A, 14B, 14C, and 14D illustrate exemplary user interfaces displayed to a user as part of a computerized pipeline system for making eye health assessments, in accordance with some embodiments of the present disclosure.

FIGS. 15A, 15B, and 15C illustrate exemplary user interfaces displayed to a user as part of a computerized pipeline system for making eye health evaluations and recommendations, in accordance with some embodiments of the present disclosure.

FIG. 16 illustrates an exemplary user interface displayed to a user as part of a computerized pipeline system for making eye health evaluations and recommendations, in accordance with some embodiments of the present disclosure.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the disclosure to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

An eye health assessment system is capable of making inferences regarding eye health of a patient based on data regarding the patient's behavior with an electronic device. The system is also capable of making recommendations to the patient or to a third party with regard to the patient, or determining an overall eye health score based on the inferences drawn from the behavioral. The inferences regarding eye health may be made by a machine learning system that trains one or more machine learning models in accordance with historical eye health data from an aggregated set of patients. These inferences may also be made by a specialized computing system based on one or more algorithmic or rules-based analyses. The system may output scores and/or inferences that may be utilized in various analytics and visualizations, which may be turn be made available to the patient or a representative of the patient (e.g., a parent).

The system may also be connected via a communications network to third party systems, for example a health system belonging to a medical practitioner, an e-commerce system, or a social media or shared system. The system may be capable of establishing a communication channel between the system and one or more health care professionals, such as eye care professionals, or additionally or alternatively, facilitating communication between the patient and such health care professional(s). The system may be capable of facilitating a healthcare appointment for the patient with a healthcare professional (e.g., for assessment of an eye health condition or potential condition), whether an in-person appointment at a physical office, or a telemedicine appointment. Communication with a health care provider may be provided at the office level, and identification of an appropriate individual provider (or more than one) can be performed in an automated manner, initiated by the performed assessment of the user's behavioral activity and not specifically (that is, not solely or by necessity) initiated by user request. In this manner, the system may act beyond only notification or recommendation to the user, and may additionally provide an automated pipeline for the user from assessment to in-person appointment.

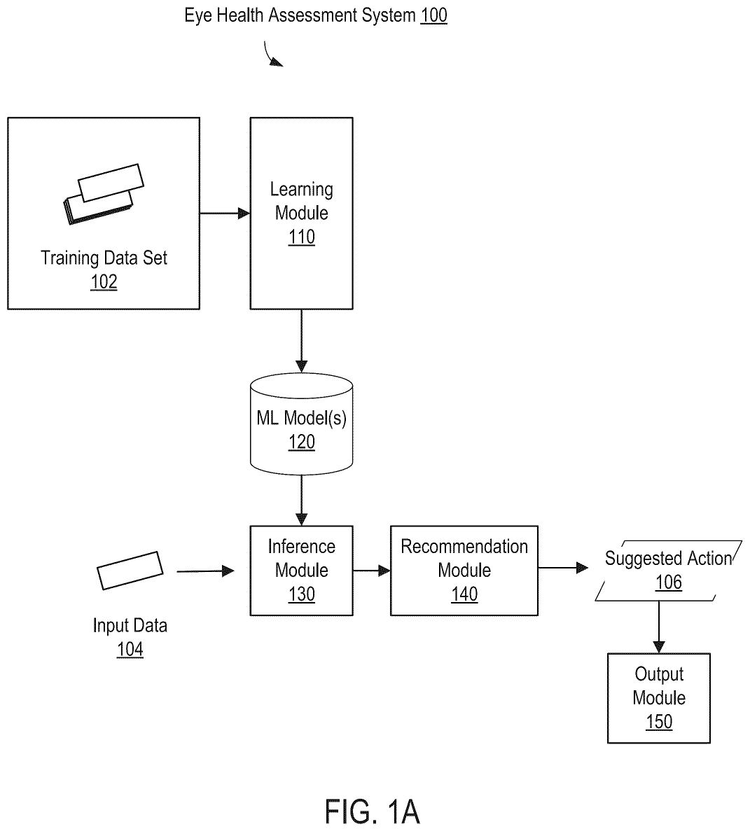

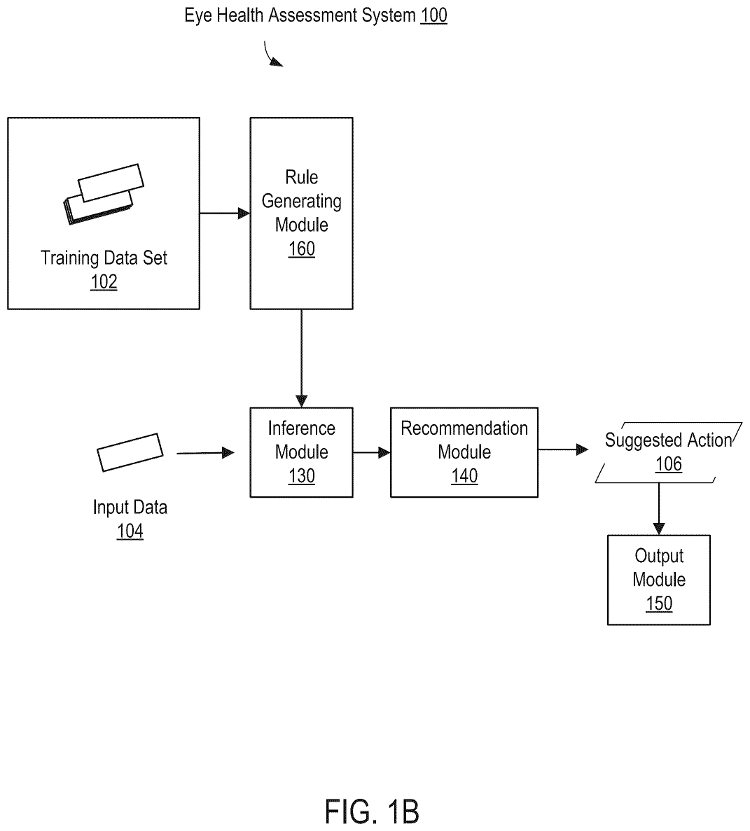

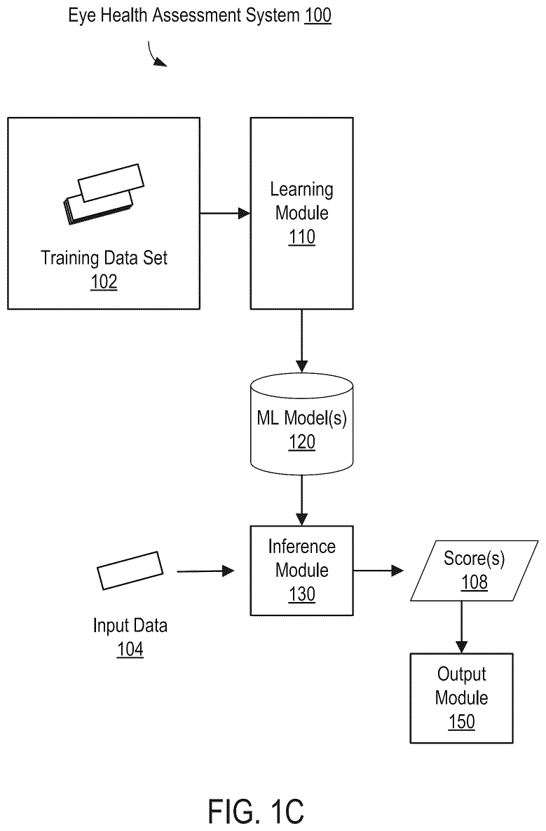

FIG. 1A illustrates an example embodiment of an eye health assessment system 100 for characterizing eye health of a user. A user may be any human, and in some embodiments may be a pediatric patient. In some embodiments, the user may have been diagnosed as having or being at risk of an eye health condition. In some embodiments, the user may be a patient of an eye health professional, or the parent or guardian of such a patient. As illustrated in FIG. 1A, a learning module 110 receives a training data set 102 and applies one or more machine learning algorithms to generate one or more machine learning models 120.

The training data set 102 may include at least medical data relating to a plurality of patients (at least one or more patients, the term patient being used here even though the data may not be strictly limited to individuals who are active patients of an eye health provider) and may include data such as but not limited to clinical trial data, health record data, and/or diagnosis data, and may additionally include purchase history data relating to the plurality of patients. The training data set 102 may include other data such as behavioral data relating to the plurality of users' (or a subset thereof) interactions with one or more electronic devices, including for instance, for any respective user, a distance between the user of a particular electronic device and the screen or display of that electronic device, the brightness of the screen or display of an electronic device, and/or length(s) of time(s) the user interacts with the device or performs a particular type of task on an electronic device. It will be understood that while the singular word “device” is used herein and throughout the disclosure, data may be collected from and/or regarding one or multiple electronic devices, collectively and/or individually. The training data set 102 may include other data such as environmental data relating to the environmental conditions of the location of plurality of users (or a subset thereof), including for instance whether they are in an indoor or outdoor environment (or another categorization or classification of a user's environmental or physical space), the lighting conditions of their environment, the time(s) of day the measurement (such as screen usage) was made or data was recorded or collected, and so on. The training data set 102 may also optionally include general image data containing images of the user and/or the user's eye, including for instances images taken with a camera function of the user's electronic device (e.g., a smart phone camera), images of one or more other individuals' eyes, and/or images (and/or other image-related data) taken from medical imaging equipment in the office of (or otherwise provided by or in support of) an eye care professional (ECP) or eye health care provider. These additional training images may be annotated or unannotated. The training data set 102 may be stored in a local storage medium, cloud-based storage, or a combination thereof.

The learning module 110 applies one or more machine learning algorithms to the training data set 102 to generate the one or more machine learning models 120. Generally, the learning module 110 may operate an offline manner. However, in some embodiments, online learning techniques may be employed to update the machine learning models 120 (periodically and/or in real-time/near real-time) as new data are acquired and added to the training data set 102. In some embodiments, the machine learning models are updated in relative real-time to the acquisition of new training data from training data set 102.

The machine learning models 120 may include one or more of a plurality of machine learning models. Machine learning models 120 may include at least a first machine learning model(s) capable of taking in a user data regarding a plurality of users (each of the “users” being understood here as a patient or other individual interested and/or seeking information about eye health), and segmenting the plurality of users into patient subgroups that are clinically distinct and associated with meaningful differences with regard to one or more eye health conditions. The segmentation of the users may be based on a population having an eye condition, such a myopia or pediatric myopia, though the segmentation is not strictly limited to an eye condition or any particular condition. In some implementations, the segmentation may alternately or additionally be made based on consumer personas, e.g., a population of users that use (or have interest in) a similar consumer product or product group.

Machine learning models 120 may also include a second machine learning model(s) capable of taking in data regarding an individual user and classifying the individual user into one or more of the patient subgroups identifying by the first machine learning model.

The machine learning model(s) 120 can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model(s) 120 is trained using supervised learning algorithms, unsupervised learning algorithms, semi-supervised learning algorithms (e.g., partial supervision), weak supervision, transfer, multi-task learning, or any combination thereof. In particular embodiments, the machine learning model(s) 120 is trained using weak supervision techniques. In particular embodiments, the machine learning model(s) 120 may be trained using one or more deep learning algorithms.

In various embodiments, the machine learning model(s) (and its submodels) has one or more parameters, such as hyperparameters or model parameters. Hyperparameters are generally established prior to training. In various embodiments, hyperparameter optimization (e.g., grid search) is performed via cross validation. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, node values in a decision tree, and coefficients in a regression model. The model parameters of the machine learning model are trained (e.g., adjusted) using the training data to improve any inferential and/or predictive capacity of the machine learning model(s).

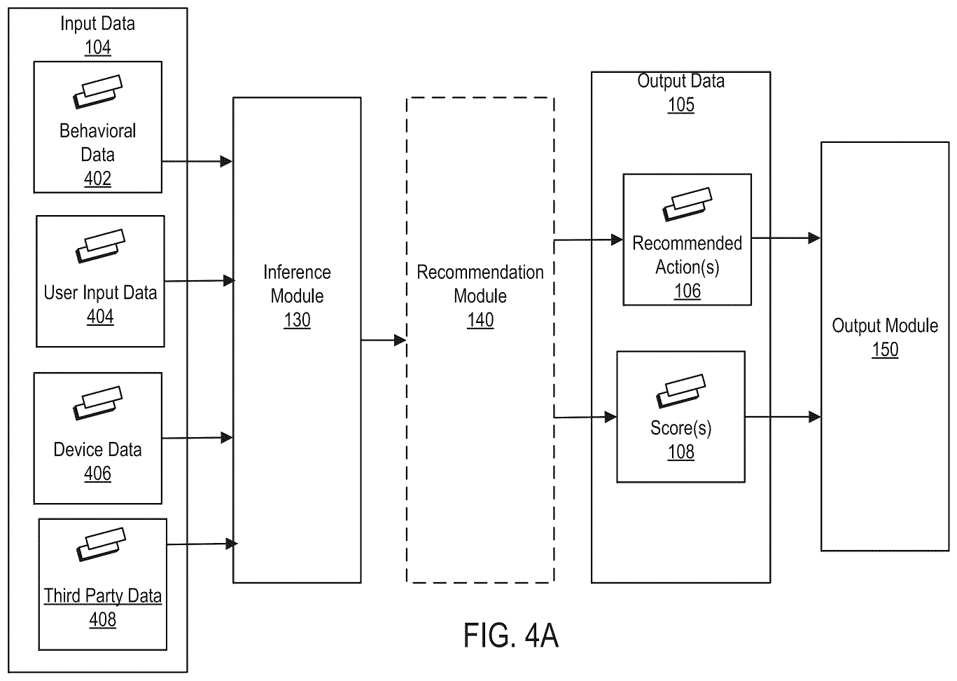

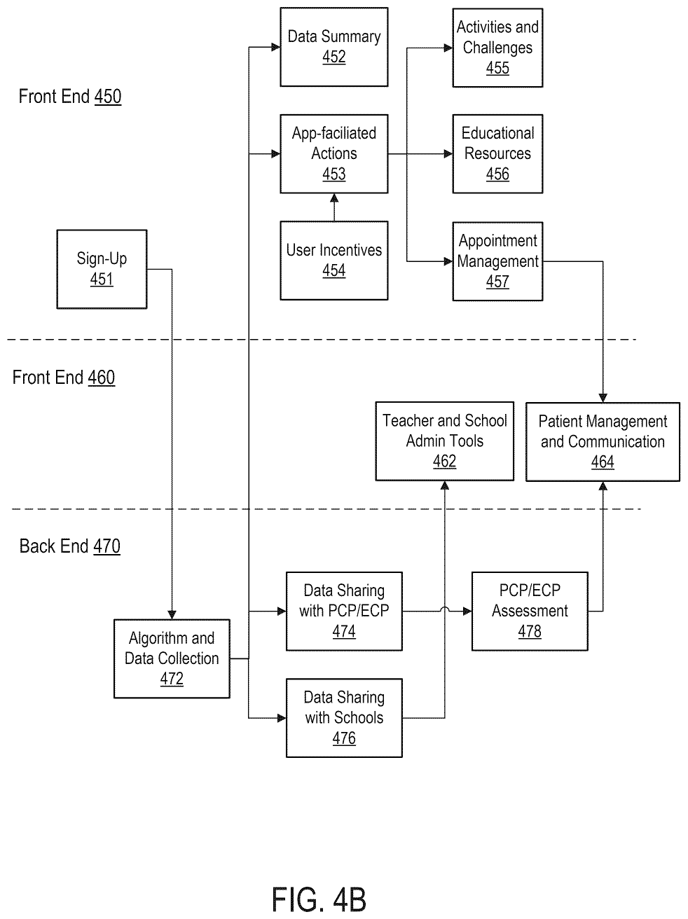

The inference module 130 receives input data 104 and applies the one or more machine learning models 120 in an inference algorithm to infer one or more actions for system 100 to take in response to any or all of the input data 104. With reference to FIGS. 4A and 4B, input data 104 may include a variety of types of data relating to an individual user, such as but not limited to behavioral data 402, user input data 404, device data 406, and/or third party data 408.

Behavioral data 402 may include, for instance, data regarding a user's use of an electronic device (such as but not limited to a smartphone, tablet, computer, handheld or tabletop device with a screen, and/or other peripheral or device(s)), or the user's interaction with their physical environment, including for instance a distance between the user of an electronic device and the screen or display of that device, a physical posture or positioning of the user, the brightness of the screen or display of the device, the length of time the user interacts with the device or performs a particular type of task on the device, a measurement of time (a number of minutes/hours, percentage) spent in an outdoors space (or a comparative analysis of indoor/outdoor time), sleep pattern data, or the like. In some embodiments, any data collected regarding physical environment (or any calculation based thereon) may be made with regard to in the user's geographic location, time of day/season/year, weather conditions, and/or other factors that may be relevant to a determination of sufficiency or character of environmental condition. In some examples, behavioral data 402 may be collected by or from one more peripheral wearable devices such as jewelry, accessories, medical devices, and clothing or elements of clothing, an implanted or transdermal device, wearable smart tags or computers/devices, or the like. As one example, behavioral data 402 may be collected by or from a smartwatch or other wrist-worn device or other fitness tracker that is capable of sensing user activity. Behavioral data 402 may be additionally or alternatively collected by or from other user-worn devices such as smart glasses, jewelry, VR headsets, smart jewelry, web-enabled glasses and Bluetooth headsets, or the like. Where behavioral data 402 is collected by or from a wearable (or other peripheral) device, such device may be transmitted to system 100 via one or more computer networks, or may be pulled or requested by system 100 from one or more intermediate and/or third party servers, such as a remote server capable of synching with a wearable device. Behavioral data 402 may additionally or alternatively be collected through user or third-party input, sensed data (environmental measurements, movement data, video, image and/or audio data or other sensed (or calculated data) collected from sensors located outside the body or otherwise not worn by the human body, and/or data stored on one more servers or other local or remote data sources.

User input data 404 may include, for instance, any or all of user survey data, prompted question/answer data (e.g., via a UI), or other data input by the user (or a guardian/representative thereof) via the user's device such as but not limited to a smartphone, tablet, computer, and/or peripheral, a third party system or platform (e.g., a website or app), regular log data entered by the user (question/answer and/or freeform text or audiovisual), user biometric data, or other data entered by the user through a screen or input peripheral of the device (e.g., camera image data, voice/video data, keypad/controller entry), or the like.

Device data 406 may include but is not limited to device sensor data such as light or ambient light data, gyroscopic data, temperature data, positional data, location data, and the like, and/or information about the device(s) itself (device type, IP address, registration information, etc.), app data, smartphone or device usage data, or any combination of any of the foregoing, or any data calculated from any data sensed or measured by the device. Device data 406 may additionally include information about how the device is being held and/or manipulated by the user.

Third party data 408 may include, for instance, data obtained from a third party system or device, such as e-commerce data (user's (or user-related) purchase data, desired or suggested purchase data, order history data), social media data, data collected regarding the user from a healthcare provider (e.g., an ECP, physician, or other health care provider system) such as patient data, clinical data, virtual consultation data, and/or appointment data. In the case that third party data 408 contains data from a health care provider, it may in some embodiments be obtained via one or more APIs or other integrated software connecting system 100 to a patient management system via one or more networks (including without limitation secure networks or storages). In some instances, third party data 408 may include data collected from an automated or computerized eye health assessment tool, such as a tool for diagnosis and/or assessment of progression of myopia, presbyopia, glaucoma, or another eye health condition, such data being obtained from one or more local or remote data sources. The patient's use of such an automated tool may be conducted via the health care provider (e.g., in an eye care provider's office) or via one or more remote solutions (e.g., those implemented in another third party facility, via the user's phone or device, via an internet-based application, or another application). The information from the health care provider may be any one or more of: patient records, measurements, risk assessment score(s), diagnosis, image data (e.g., OCT or other standard eye health images), data indicating the progression of an eye condition and/or eye health, doctor's assessments, notations and/or textual data, and/or non-eye related healthcare data that may be relevant to an assessment of an eye health condition (e.g., underlying health conditions). In some instances, input data 104 may include benchmark patient data from an eye health care provider and/or other clinical data repository, and may include without limitation historical data of the individual or aggregated benchmark data of a plurality of patients or users.

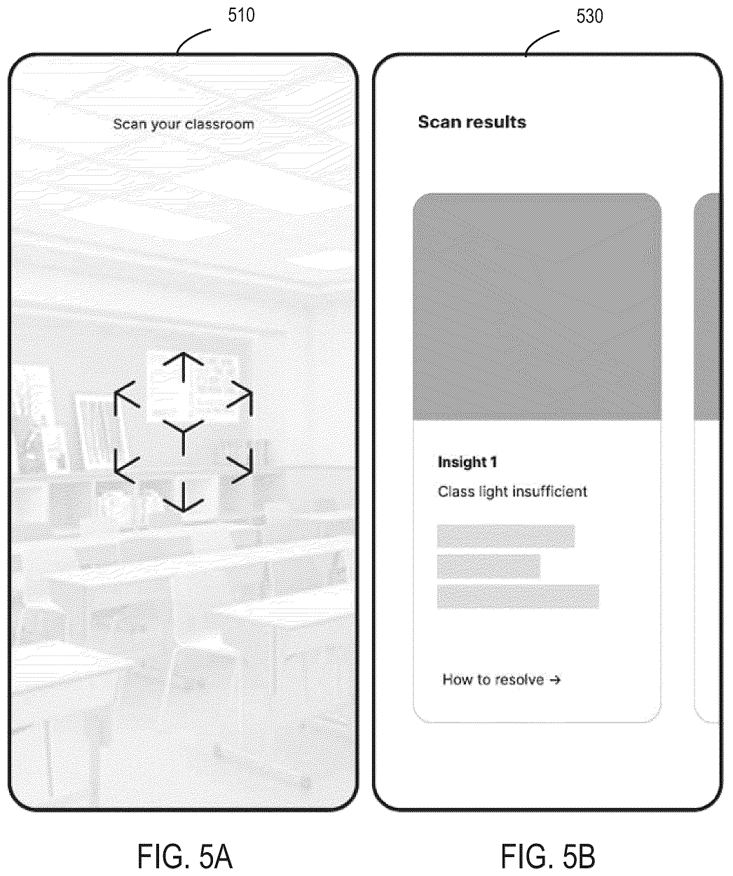

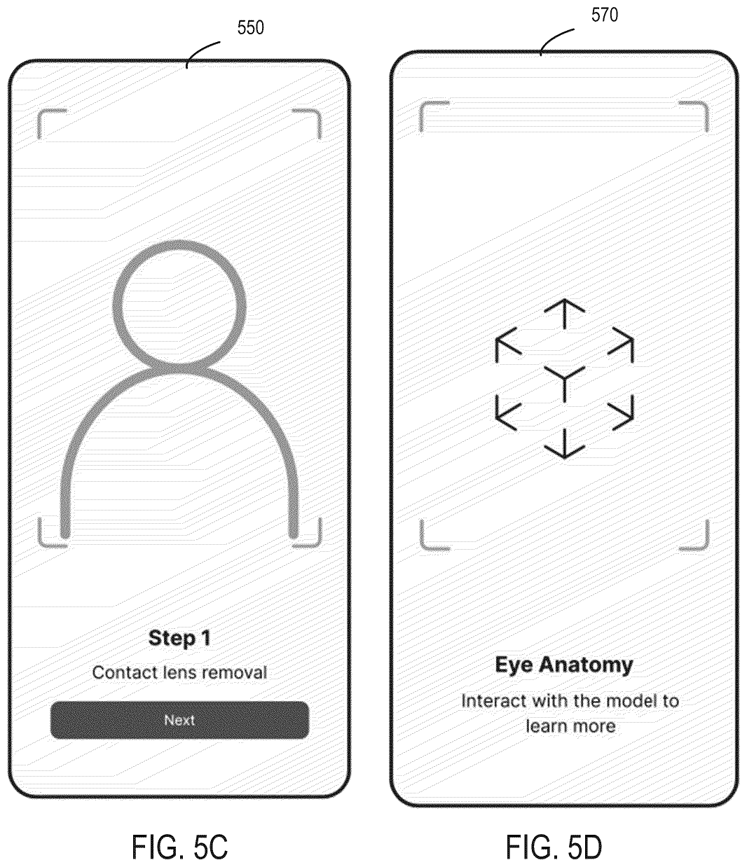

Input data 104 may be obtained through any a variety of sources; for instance, environmental lighting condition data may be obtained through one or more device sensors automatically (as device data 406), or with reference to FIGS. 5A, 5C, and 5D though an intentional collection and sharing of information by the user through one or more camera and/or audiovisual devices (user input data 404), whether integrated with or communicatively coupled to a user's device and/or system 100.

FIG. 5A illustrates an example of a camera-enabled functionality wherein information regarding the user's environment can be taken in through a visual and/or auditory scan of the physical space using the device's camera (image 510 depicting the device's screen). FIG. 5B illustrates a possible exemplary output of a scan as depicted in FIG. 5A, wherein screen 530 displays deficiencies in the environment that may be relevant to the eye health of the user (e.g., class light insufficient). Similarly, the screen may indicate deficiencies in the environment that prevent completion of the scan. The outputs and/or insights generated by such a scan may be used to improve the quality of an environment. For instance, the scan and insights depicted in FIGS. 5A and 5B might be used by a teacher to improve the quality of the classroom environment.

FIGS. 5C and 5D illustrate an example of a camera-enabled functionality (using, e.g., augmented reality or AR) wherein the user's eye may be scanned, and information regarding eye anatomy, eye health, vision maintenance, and/or contact lens usage can be presented to the user for, e.g., an educational purpose.

In some instances, input data 104 may include data calculated, aggregated, and/or derived by system 100 from any of the behavioral data 402, user input data 404, device data 406, and/or third party data 408. For instance, system 100 may generate and/or capture, via computational methods or aggregation/reports from input data 104 information such as scoring data, visual acuity test (VAT) data, augmented reality (AR), mixed reality, or virtual reality (VR) data. In some instances, system 100 may include computing modules configured to generate a score by comparing a subset of input data 104 to benchmarked data stored internally in or externally to system 100.

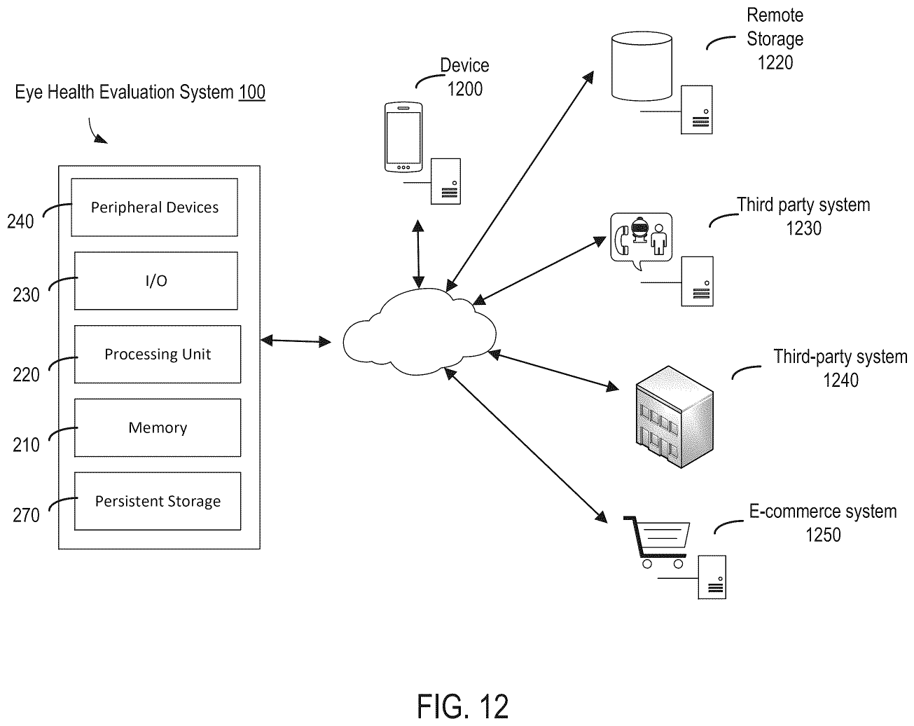

FIG. 12 further illustrates how system 100 may, in some instances, be communicatively connected to one or more third party systems. This may, in some embodiments, facilitate the collection and/or receipt of third party data 408. For purposes of illustration, system 100 may access data from a user's device(s) 1200, which may be, without limitation, a smart phone or other mobile or cellular-enabled device, a tablet, a personal computer or similar (e.g., desktop or laptop), a gaming console, a smart device (e.g., smart TV), a camera-enabled device, a console device, a specially-designed computing device, or any other device capable of taking in information from the user and connecting to a computer network for transfer of data. It will be understood that the user may be the patient or a representative of the patient (e.g., parent, guardian, health care provider, and so on). Third party systems may include, without limitation, any remote data store 1220, any third party system 1230 such as, e.g., a social media platform, smartphone apps, game platforms, educational platforms, and the like), any third party system 1240 (such as, e.g., an ECP, primary care physician (PCP), or other health care provider system or related service (including patient management, appointment/schedule management, and/or communication systems), a school or university computer system (such as via one or more teacher or admin tools), any e-commerce system 1250, or any other third-party system as variously described herein, or any combination of any of the foregoing. The third party system 1240 may be an automated service or tool (e.g., an AI-based scheduler, chatbot, assistant, or the like). Communication with third party system 1230, 1240, 1250 may additionally or alternately involve telephonic/voice-based, text-based, structured data exchange, interaction with websites/forms, web-based (e.g., TCP/IP) exchanges, or alternate communication methods with human or automated agents.

Communications performed by output module 150 are also further described with reference to 4B. In particular, visual and/or user interfaces may be put into effect by one or more front end (user interface) functionalities 450 (with the user) and 460 (with third parties) and supported by one or more back end functionalities 470 (occurring within a server-side implementation of system 100). A user or patient may, via a user interface, sign up with the system 100 (sign up block 451), wherein they would enter personal informal, login information, and so on. Data entered as part of the sign-up process is processed and/or stored by Algorithm and Data Collection functionalities 472. Functionalities 451 and 472 may work in tandem (or communicate with each other) to obtain from a user (via one or more user interfaces) input data 104 from the user as described above and otherwise herein. Data collected may be used in a variety of communications and other interactions with the user and third-party entities as described herein.

Recommendation module 140 generates output data 105, which is output to the user (and/or other third party entities) by output module 150. With reference to FIG. 1A, FIG. 4A, output data 105 may include a recommendation to the user of an action (or suggested action) 106. Output data 105 may also include other content (or may be used to generate other content) for display to the user as described further herein. With reference to FIG. 1C and FIG. 4A, output data 105 may additionally or alternately include one or more scores 108 characterizing health of a patient. The one or more scores 108 may be any one or more of: a numerical value, a textual description, a percentage, a risk value, a value associated with any standard medical scoring systems, a binary value or classification indicating the presence or absence of a condition (or another indication of a likelihood thereof), an image/graphic, chart or visual indicator, or any similar signifier of eye health. The score 108 may indicate an eye health of the user, but in alternate implementations, a score reflecting a different vertical of health (such as cardiovascular health) or overall patient health may be generated.

In some embodiments, both a recommendation for an action 106 and a health score 108 may be generated. In some implementations where only a score 108 is generated, recommendation module 140 may not be present in system 100, or may not generate any output, such that inference module 130 may generate a score 108 and/or other output data 105.

In some embodiments, score 108 is not generated based on use of a particular electronic device, but instead represents the user's aggregated or holistic use of and across any of multiple electronic devices (e.g., smartphone, tablet, computer, smart device, gaming system, TV, and the like), thus providing a more comprehensive view of device usage.

In generating a recommendation 106 or score 108, the inference module 130 (and/or recommendation module 140) takes input data 104 and applies one or more machine learning models 120 to determine whether the user may appropriate be classified into one or more patient subgroups. This classification may in some instances be based on a horizontal view of the user's data over time, such that input data 104 may take in historical input data 104 to infer a change in the user's condition. The classification additionally may not be final or permanent, such that the user's classification may change over time or from day-to-day or measurement-to-measurement. For instance, where the classification is based on an environmental condition (such as whether there is sufficient indoor lighting), a change in the user's location or in the environment may lead to a re-classification.

The output module 150 obtains the recommendation(s) 106 and/or the score(s) 108 and may generate various analytics, user interface displays, or other outputs relating thereto. These may be understood with reference to FIG. 4B as data summary 452 transmitted to a user via one or more user interfaces. For example, the output module 150 may generate visual representations of the raw scores, various visualizations, charts or graphs characterizing the scores, and/or various recommended treatments relating to the scores.

Such visual representations may be displayed e.g., to the user, a medical practitioner, and/or a desired third party (e.g., a parent or guardian) via a computer display screen, a network accessible interface or cloud-based platform, or another medium. In some embodiments, a recommendation may take the form of a report, video/audio content, notice, or alert to the user, as an answer to a user input question or as additional information for the user's benefit. While the term “visual representations” or “display” may be used herein, it will be understood that, in some embodiments, output module 150 may, in addition or as an alternate to visual representation(s), provide recommendations or representations in the form of (or including) auditory, haptic, and/or other non-visual alerts, notifications, or other feedback. Further, in some instances, output module 150 may be configured to determine which form of display or presentation (or combination thereof) should be used to present information to the user based at least in part on recommendation 106 or score 108. As just one example, by way of illustration and not of limitation, in a case where recommendation 106 and/or score 108 indicates that a user may have low visual acuity, output module 150 may, in addition to a visual representation displayed on a computer screen, transmit to the user a non-visual (e.g., audio or haptic) option for receiving recommendations or data representations.

In further embodiments, the output module 140 may display recommendations 106 and/or output scores 108 or related data associated with a patient that is tracked over time to characterize progression (or other assessment) of an eye condition.

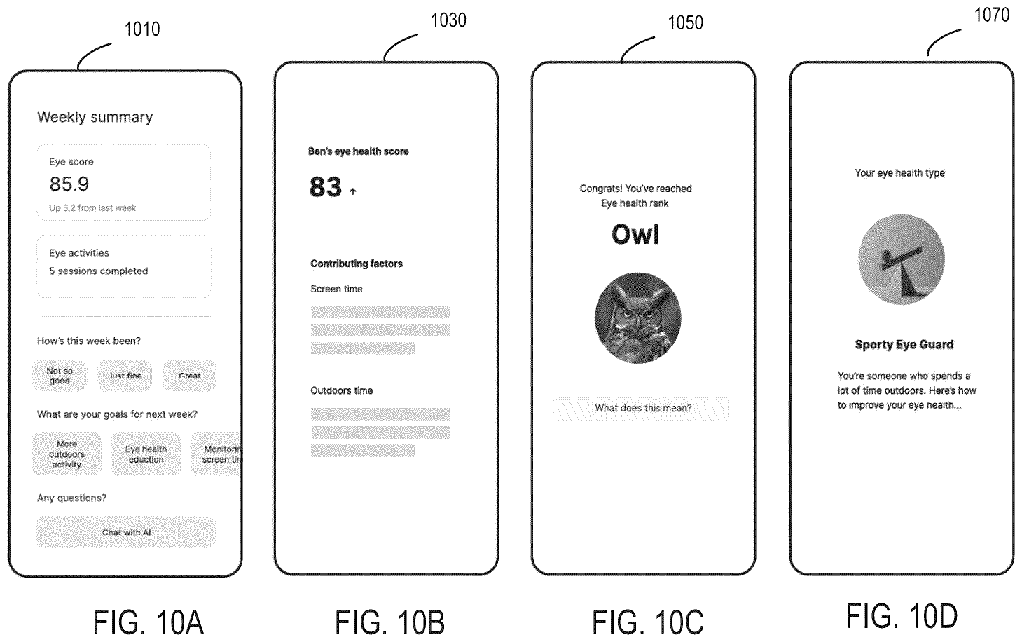

Furthermore, the output module 140 may present the scores 108 as an overlay on a display or in combination with other information on a user interface (FIG. 10A-10D). The score may be displayed as a numerical score (FIG. 10A or 10B) as a periodic (daily, weekly, etc) summary or overview (screen 1010) or single score and explanation/detail (screen 1030), a binary indicator, and/or with or as a classification or label designed to be meaningful to the user (e.g., FIG. 10C, 10D). Such a classification, rank, and/or label can be assigned to the user (an animal classifier in screen 1050, a “type” in screen 1070, or other type or grouping or label) to engage and motivate the user, or to provide an easily understand method to track progress/health. This may additionally or alternately include but is not limited to AR/VR content presented over an image or video.

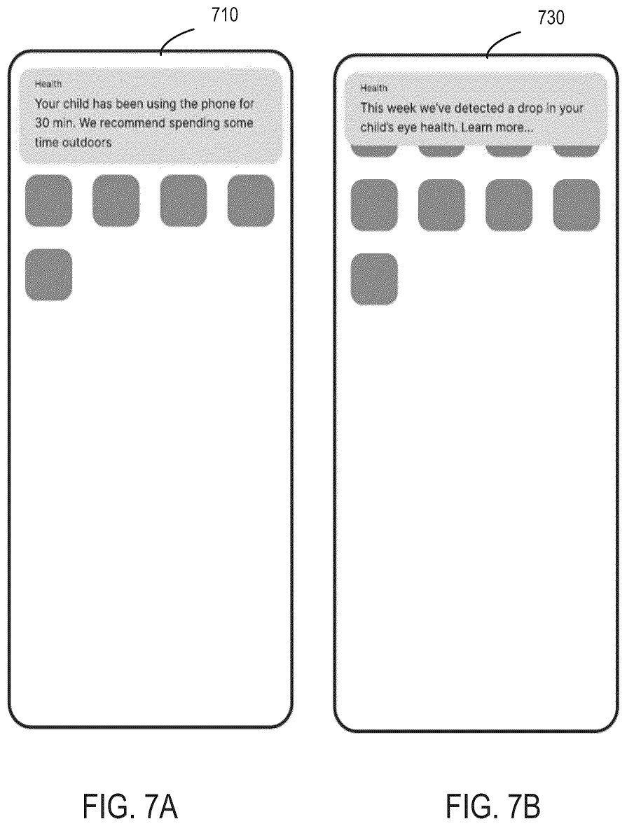

A displayed representation of the recommendations may include information regarding the environment, e.g., an evaluation of the sufficiency of light as in FIG. 5B, or an alert to a change of behavior as in FIG. 7A or change in overall eye health (FIG. 7B) or other types of deviation.

Output module 150 may additionally or alternatively present the recommendations to the user by initiating one or more actions that require interaction with the user, which may be understood with reference to FIG. 4B as app-facilitated actions 453 via one or more user interfaces. The displayed representation of the recommendations may include a discussion of the user's eye health, including for instance recommendation to contact a health professional, an in-person or AI-assisted chat function. In some implementations, the recommendation may include information that the user should convey to their PCP/ECP in their next appointment. In some instances, the representation may include content to promote a healthy lifestyle. In some instances, the displayed representation may include the presentation one or more software-enabled features such as a visual acuity test (VAT), virtual consultation with an eye health provider, or patient management features.

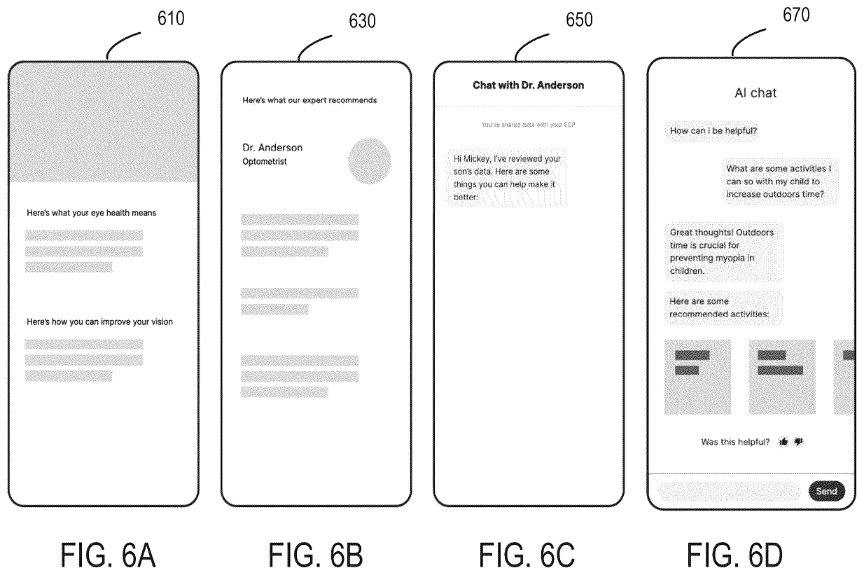

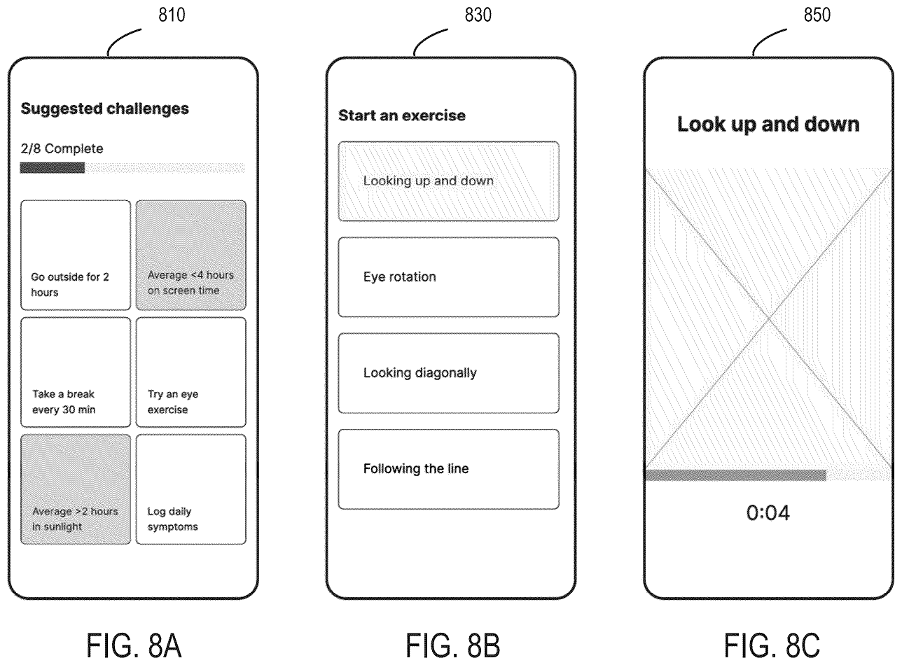

In some instances, the displayed representation may include educational material (educational resources 456, also displayed in FIG. 6A as screen 610 or similar) and/or other content tailored to suit the attention, engagement and/or understanding of users of one or more of various ages/classifications, e.g., child and adult users (activities and challenges 455 such as games or gamification functionalities and/or user incentives 454 such as discounts, codes or specials, related deals and products). FIGS. 8A, 8B, and 8C depict just one example of such activities or challenges, though any appropriate challenge directed to eye health, or a factor relating to eye health or overall health may alternate implemented. Challenges may be selected that are targeted to the user, an eye health condition, a group or classification into which the user is fit, a generalized health suggestion, a selection by a healthcare professional or the user's ECP/PCP, a selected by a teacher or education partner, or as otherwise appropriate. Screen 810 depicts an assortment of exemplary challenges that the user can select. Screen 830 depicts exemplary activities under the challenge and allows the user to select an activity. Screen 850 depicts an example of a screen guiding or assistance performance.

In some cases, system 100 may be connected to a variety of third party health care systems, and rather than (or in addition to) a displayed representation of the recommendation(s), the output module 140 may function to connect with an ECP/PCP system to facilitate the scheduling of an appointment, an online vision screening or exam, a virtual consultation, and/or a chat or other communication with the ECP/PCP (e.g., via a portal or chat feature, email, or the like). While the term ECP, PCP, or ECP/PCP are variously used herein, it will be understood that the description is not so limited and any health care provider system (or relevant related system, e.g., insurance system) may be used in an implementation under the systems and methods described herein. In this regard, system 100 may provide to the user one or more mechanisms with which to communicate with a ECP/PCP, including appointment manager 457. System 100 may also provide one or more mechanisms (user interfaces) for patient management and communication 464 to an ECP/PCP, or a common portal/site for multiple/ECPs/PCPs. A user may be presented with any of recommendation to contact a health professional (FIG. 6B, e.g., screen 630 or similar), an in-person (FIG. 6C, e.g., screen 650 or similar) or AI-assisted (FIG. 6D, e.g., screen 670 or similar) chat function, and/or or one more interfaces for actually scheduling a remote or in-person appointment.

In some implementations, the user may have already input contact information regarding one or more preferred health care providers (e.g., ECP, PCP, or other provider), and in such scenarios, system 100 may default to contacting the preferred health care provider. In other scenarios, where the user has not indicated any preferred provider, or has provider multiple providers without an indication of preference, the system 100 may comprise one or more functionalities capable of identifying an appropriate ECP/PCP provider. In some cases, where a default provider has been identified by the user and the user has given appropriate permission, the system 100 may create an appointment and/or visit schedule for a user with the default provider without initiation and/or additional required action (e.g., approval) by the user, that is, in an automated fashion. Data sharing with one or more ECP/PCP systems may be facilitated by back end functionalities 474. Additionally, ECP/PCP assessment functions 478 may be configured to identify an appropriate provider of the user. ECP/PCP assessment block 478 may consider factors such as (1) geolocation based on a location of the user's device (device data 406), home address, and/or other identified address and the location of one or more known or preferred ECP/PCPs, (2) assessment of what services or specializations a particular ECP/PCP provides or other qualities such as number of providers, insurance compatibility with user input, and so on (such provider information being stored as third party data 408 or obtained in real-time/near real-time as a query to the provider), (3) assessment of the user's provider history (medical records) or input data 404, (4) participation of the provider in one or more programs, and/or (5) assessment of other information contributing to the appropriateness of a provider, e.g., if a relative/parent has entered provider information (where prior permission has been granted to access the other respective user). In some circumstances, ECP/PCP assessment functions 478 may implement an AI model or rules-based model to weigh factors (such as items (1)-(5) above) or other relevant considerations so as to connect the user with a calculated “best” provider for their circumstances or a ranking of providers. In other implementations, PCP/ECP assessment functions 478 may generate a list that is unranked, or ranked by distance, cost, or other considerations relevant to the user.

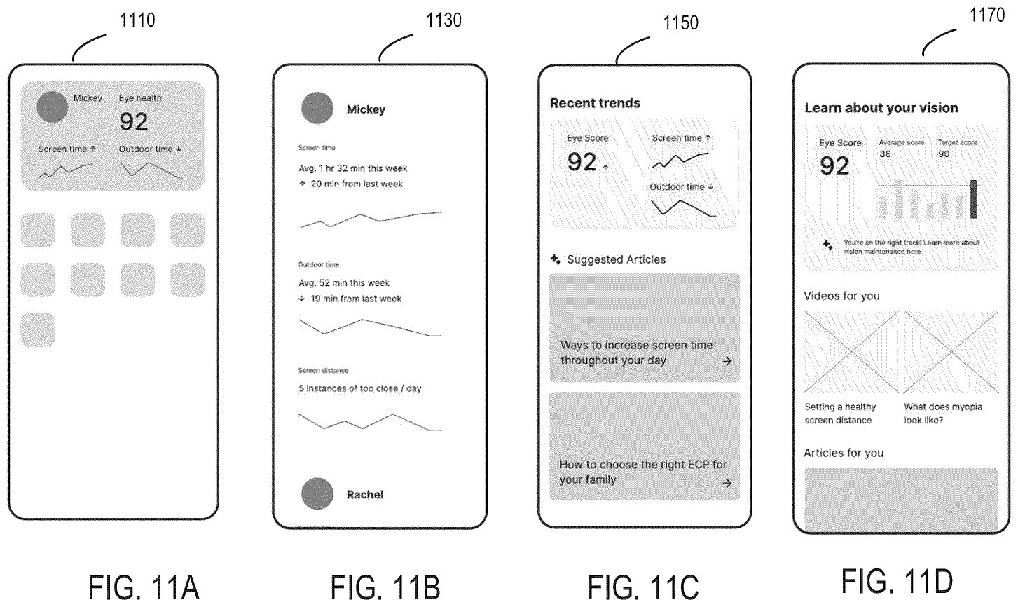

The displayed representation of the recommendations may additionally or alternatively include a conclusion or report drawn from evaluation over time. This conclusion or recommendation may be related to the user's eye health, as in FIG. 7, or FIGS. 11A-11D. FIG. 11A depicts an exemplary screen 1110 with an eye health score; screen 1110 may be understood as an overall report or dashboard for the user. FIG. 11B depicts an exemplary screen 1130 with a tracking or progress of statistics (here, for multiple users), to allow for comparison over time or comparison to others. FIG. 11C depicts an exemplary screen 1150 indicating trends of user activity. FIG. 11D depicts an exemplary screen 1170 with a user report as well as educational materials. Accordingly, a variety of user reports can be displayed on the UI of their device, and/or customized to their preference and/or level of interest or understanding.

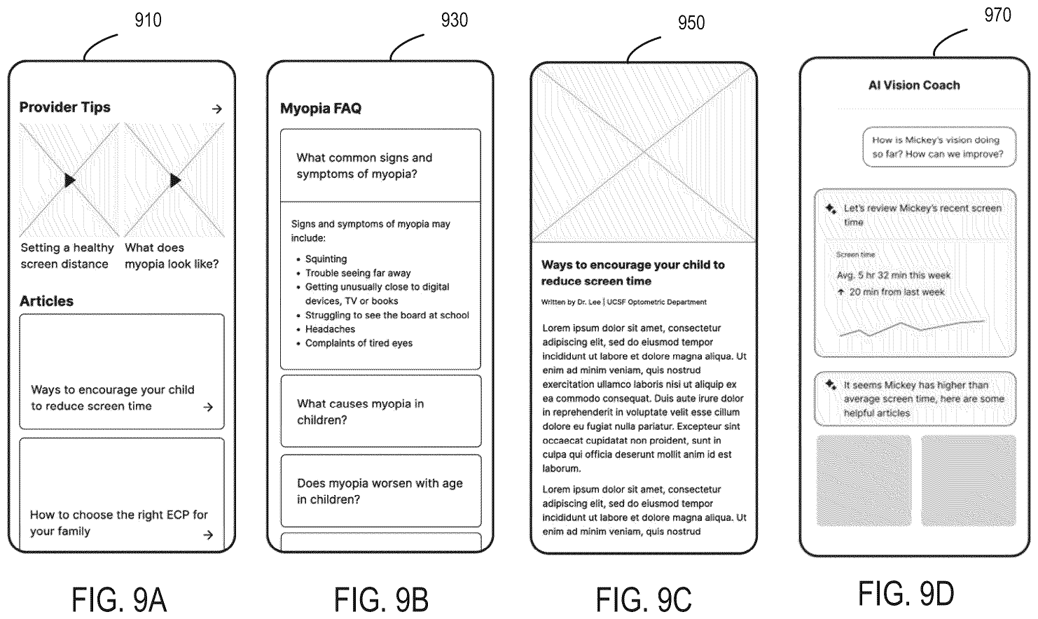

In some embodiments, treatment plans, tips, educational content (e.g., teaching or education tools), or suggestions for change in activity relating to eye health may plans may be automatically recommended based on tracked progression and may be displayed (FIG. 9A-9D). In some instances, such suggestions, tips, and/or content may be directed to an aspect of a user's health other than eye health, such as overall health, diet, cardiovascular health, or the like. As an example, tips for the user (or child/other of the user) could be displayed from a provider or scientific or other article (screen 910), as general questions directed to an eye health condition or other topic of interest (screen 930). A selection of a tip, article or topic could present a screen to the user with more detail (screen 950). In addition, a screen facilitating an AI chatbot/assistant and/or human contact could be provided for chat, audio/visual communications, or other device-based communication (screen 970). Where the system is generating educational content, it may provide such resources to the user (as educational resources 456) and additionally or alternately to a school or educational system through one or more websites or user interfaces (teacher and school admin tools 462). Where communication with an educational institution is required, the system may facilitate data sharing with schools 476.

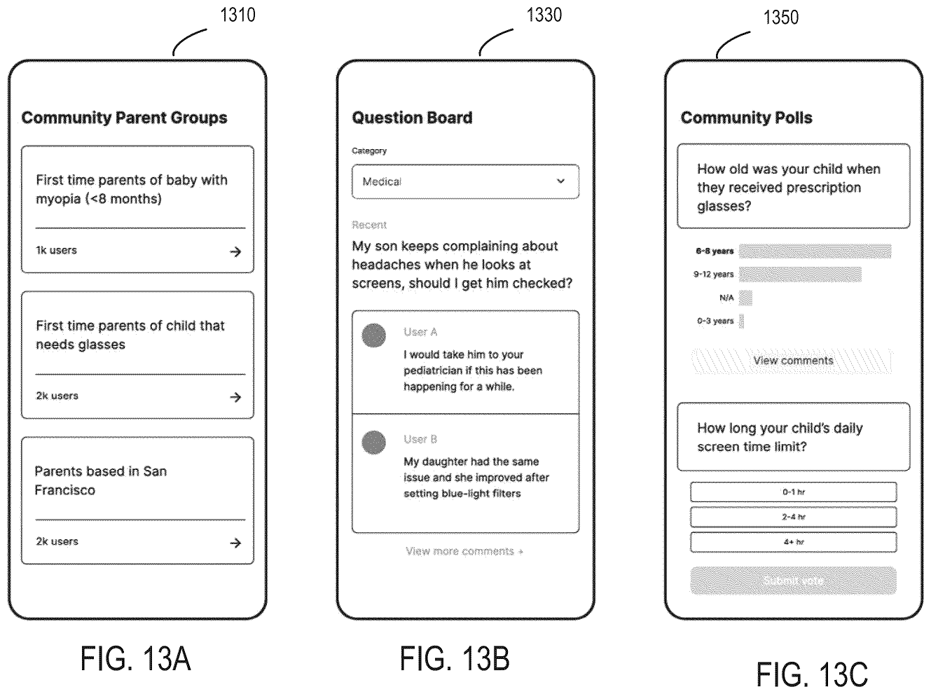

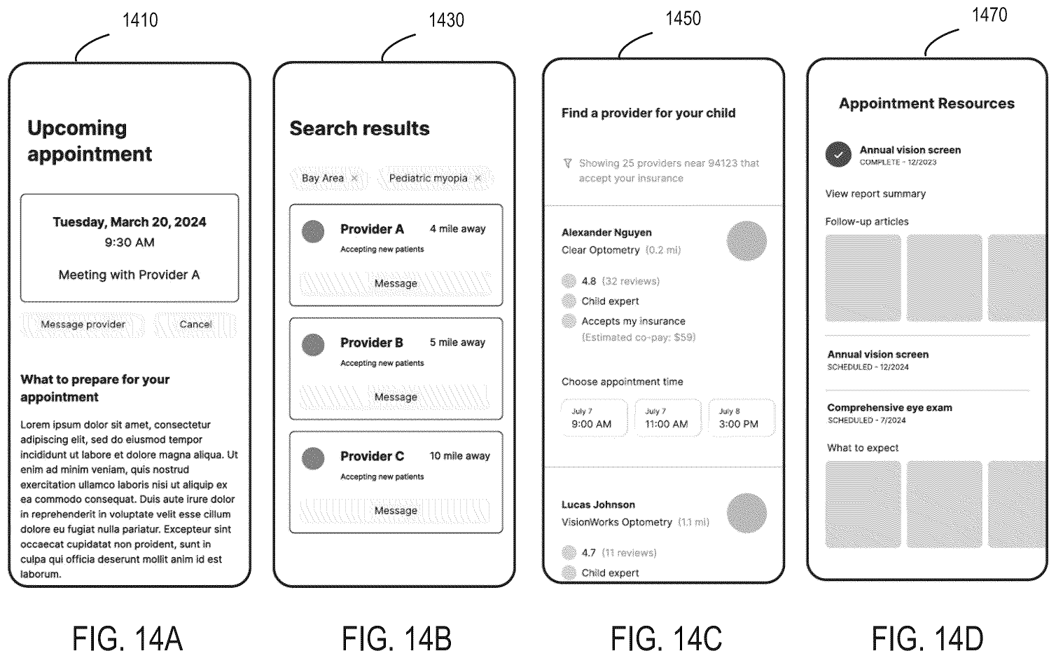

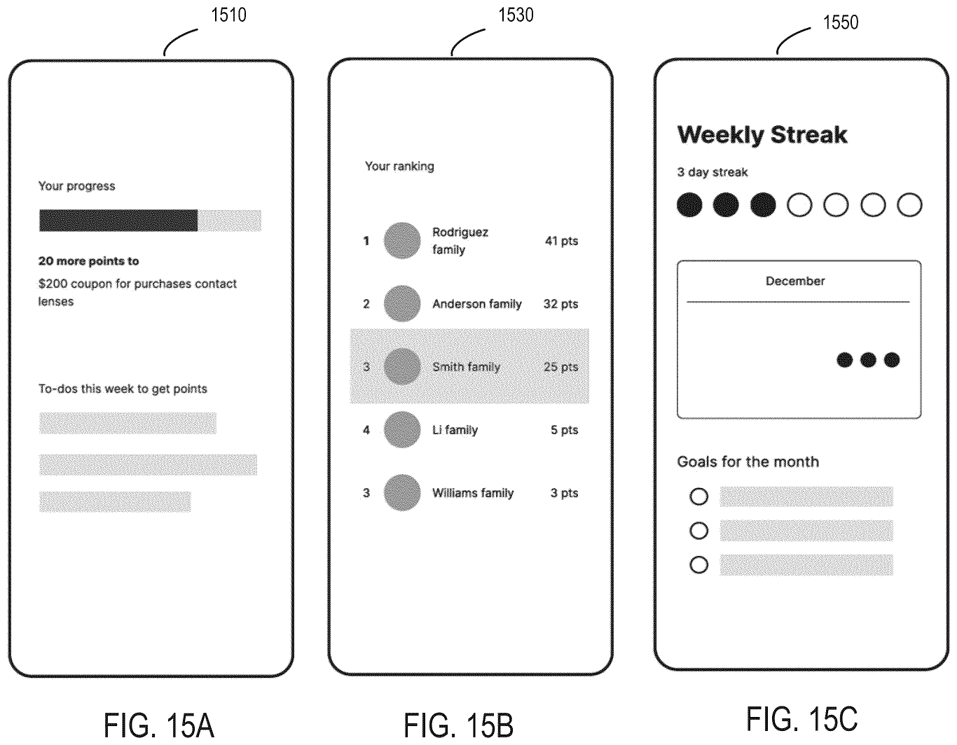

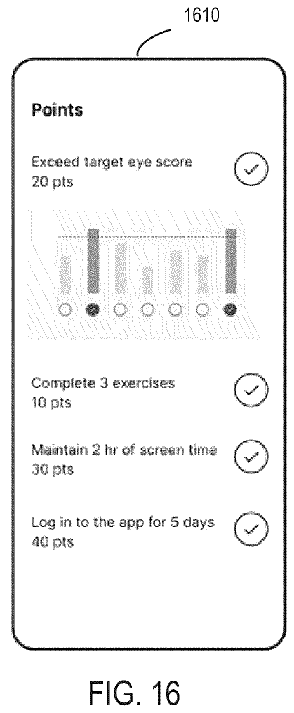

In some cases, system 100 may be connected to a variety of third party systems, and the output module 160 may allow for interactions with such systems. For instance, the output module 160 may display content from or allowing input to community groups and forums, as in FIG. 13A (exemplary screen 1310 with community parent groups), 13B (exemplary screen 1130 with question boards/forums), and 13C (exemplary screen 1350 with pools/questions for the user and/or groups of users). The output module may additionally or alternatively display information obtained from communication with health care provider systems, as in FIGS. 14A (exemplary screen 1410 showing an appointment reminder and related information), 14B (exemplary screen 1430 depicting search results), 14C (exemplary screen 1450 depicting search results for a provider by geographic and/or in some embodiments otherwise formatted generated suggested provider lists), and 14D (exemplary screen 1470 depicting appointment resources). The output module 160 may additionally or alternately display information related to purchasing and/or e-commerce platforms (FIG. 15A, depicting exemplary screen 1510 indicating progress towards discounts on relevant products), social media engines (FIG. 15B, depicting exemplary screen 1350 showing competitive rankings of the user against other individuals, teams, and/or groups), or user apps or tracking functions (FIG. 15C, depicting exemplary screen 1550 showing a screen of activity, user-set goals, and so on). In some cases, system 100 may track the user's actions against a points-based analysis (FIG. 16 and in FIG. 4B, activities and challenges 455). In some cases, the content may be displayed or delivered in a gamified manner (e.g., with badges or streaks), to facilitate engagement by the user and/or with others. This may, some cases, this point-based analysis may be provided in combination within a third-party system as described above.

Although the learning module 110 and the inference module 130 are depicted as separate components of the eye health assessment system 100, these modules 110, 130 may optionally share one or more sub modules that perform functions common to both the learning module 110 and the inference module 130. For example, the learning module 110 and inference module 130 may share various preprocessing or other functions.

In an alternate implementation shown in FIG. 1B, instead of learning module 110, a rule generating module 160 may use algorithmic rules-based methodology to draw inferences from training data set 102, without using (or in addition to using) machine learning techniques. Rather than machine learning models 120, rule generating module 160 may generate one or more analytical rules that may be applied by inference module 130.

The eye health assessment system 100 of FIG. 1 may be implemented in a computing and storage environment. Various components may be implemented using processing and/or storage devices that are co located or physically remote and coupled by a network. For example, components may be implemented using on site storage and processing and/or cloud-based systems (e.g., public, private or hybrid public private clouds). Communication between physical components may be implemented by one or more wired or wireless local area networks, one or more wired or wireless wide area networks (e.g., via wired or cellular communications), one or more peer to peer connections (e.g., over Wireless Personal Area Networks (WPANs) such as Bluetooth), and/or other communication channels for communication between different aspects of the system 100. Communication channels for the exchange or personally identifiable or otherwise confidential information may be secure and/or encrypted. In some instances, dedicated encrypted communication channels and/or digital storage may be used that are specifically configured for the transmission and/or storage of personally identifiable data and/or healthcare data. The specific functions attributed to the various modules described herein may be implemented by one or more processors executing instructions stored to one or more non transitory computer readable storage mediums.

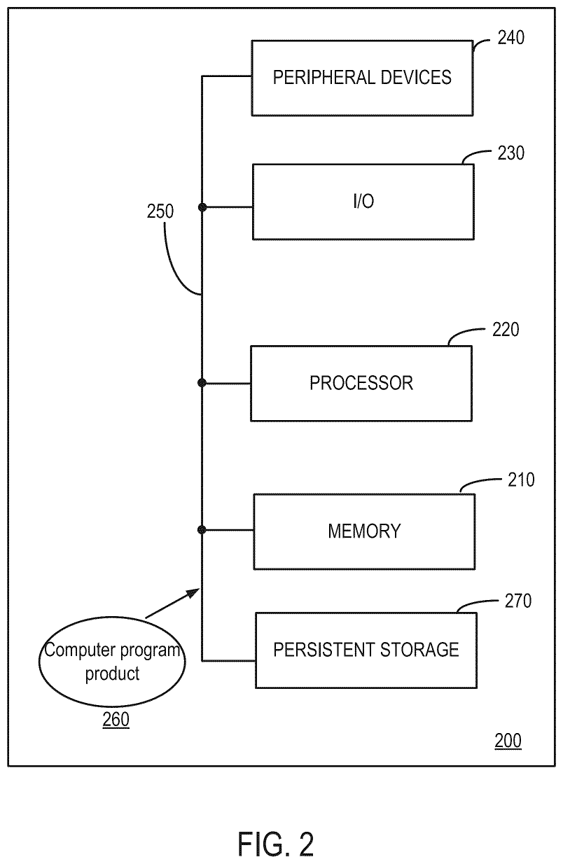

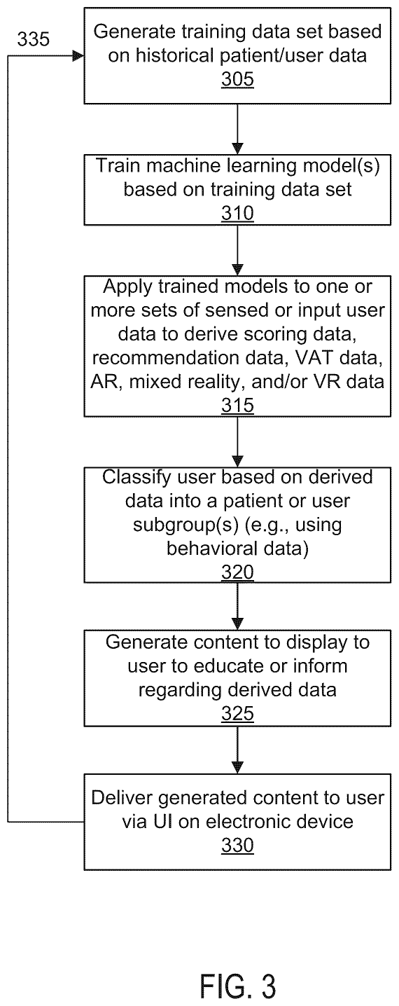

FIG. 2 illustrates one example of a computer system 200, one or more of which may be used to implement one or more of the apparatuses, systems, and methods illustrated herein. Computer system 200 executes instruction code contained in a computer program product 260. Computer program product 260 comprises executable code in an electronically readable medium that may instruct one or more computers such as computer system 200 to perform processing that accomplishes the exemplary method steps performed.

The electronically readable medium may be any transitory or non-transitory medium that stores information electronically and may be accessed locally or remotely, for example via a network connection. The medium may include a plurality of geographically dispersed media each configured to store different parts of the executable code at different locations and/or at different times. The executable instruction code in an electronically readable medium directs the illustrated computer system 200 to carry out various exemplary tasks described herein. The executable code for directing the carrying out of tasks described herein would be typically realized in software. However, it will be appreciated by those skilled in the art, that computers or other electronic devices might utilize code realized in hardware to perform many or all the identified tasks. Those skilled in the art will understand that many variations on executable code may be found that implement exemplary methods within the spirit and the scope of the disclosure.

The code or a copy of the code contained in computer program product 260 may reside in one or more storage persistent media (not separately shown) communicatively coupled to system 200 for loading and storage in persistent storage device 270 and/or memory 210 for execution by processor 220. Computer system 200 also includes I/O subsystem 230 and peripheral devices 240. I/O subsystem 230, peripheral devices 240, processor 220, memory 210, and persistent storage device 270 are coupled via at least one bus. Like persistent storage device 270 and any other persistent storage that might contain computer program product 260, memory 210 is a non-transitory media (even if implemented as a typical volatile computer memory device). Moreover, those skilled in the art will appreciate that in addition to storing computer program product 260 for carrying out processing described herein, memory 210 and/or persistent storage device 270 may be configured to store the various data elements referenced and illustrated herein.

Those skilled in the art will appreciate computer system 200 illustrates just one example of a system in which a computer program product in accordance with the disclosure may be implemented. To cite but one example, execution of instructions contained in a computer program product may be distributed over multiple computers, such as, for example, over the computers of a distributed computing network.

Instructions for implementing an artificial neural network or other deep learning network may reside in computer program product 260. When processor 220 is executing the instructions of computer program product 260, the instructions, or a portion thereof, are typically loaded into working memory 210 from which the instructions are readily accessed by processor 220.

Processor 220 may comprise multiple processors which may comprise respective additional working memories (additional processors and memories not individually illustrated) including one or more graphics processing units (GPUs) comprising at least thousands of arithmetic logic units supporting parallel computations on a large scale. GPUs are often utilized in deep learning applications because they can perform the relevant processing tasks more efficiently than can typical general-purpose processors (CPUs). Processor 220 may additionally or alternatively comprise one or more specialized processing units comprising systolic arrays and/or other hardware arrangements that support efficient parallel processing. Such specialized hardware may work in conjunction with a CPU and/or GPU to carry out the various processing described herein. Such specialized hardware may comprise application specific integrated circuits and the like (which may refer to a portion of an integrated circuit that is application-specific), field programmable gate arrays and the like, or combinations thereof. However, a processor such as processor 220 may be implemented as one or more general purpose processors (preferably having multiple cores) without necessarily departing from the spirit and scope of the present disclosure.

By means of the systems and methods described herein, a pipeline can be generated where behavioral data is collected for a potential patient, an adjustment of risk can be performed and the data can be assessed to segment the patient into appropriate classification categories with regard to risk for an eye health condition. Additionally, communication mechanisms can be put into place with an eye health (and/or general health) provider so as to provide app-through-office capability and drive users to health care providers where necessary and appropriate. In that regard, virtual enablement is provided at the office level, and identification of an appropriate individual provider can be performed in an automated manner based on the user's assessed behavioral activity (as compared to a necessary step of initiation by the user).

The disclosure includes the listed embodiments and the text and drawings appended hereto as well as any cited documents, which are incorporated by reference herein in their entirety. The description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may include a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may be architectures employing multiple processor designs for increased computing capability.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules. Similarly, the methods described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations. The one or more processors may also operate to support performance of the relevant operations in a “cloud computing” environment or as a “software as a service” (SaaS) or located in a single geographic location (e.g., within a home environment, an office environment, or a server farm). For example, at least some of the operations may be performed by a group of computers (as examples of machines including processors), these operations being accessible via a network (e.g., the Internet) and via one or more appropriate interfaces (e.g., application program interfaces (APIs).)

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

1. A method comprising:
capturing, via a device, behavioral data regarding (a) a user's use of the device, or (b) the user's interaction with the user's environment;
capturing, via the device, input data comprising one or more of: (a) user survey data, (b) user biometric data, (c) data entered by the user through a screen or peripheral of the device, and (d) device sensor data;
obtaining, from a remote system, historical data related to one or more of: (a) medical history of the user, (b) medical data relating to a plurality of patients, (c) clinical trial data, and (d) purchase data;
providing the historical data to a first machine learning model, the first machine learning model having been trained to segment a plurality of users into patient subgroups that are clinically distinct and associated with meaningful differences with regard to one or more eye health conditions;
providing at least the behavioral data and the input data to a second machine learning model, the second machine learning model having been trained to classify the user into one or more of the patient subgroups; and
performing an action in response to the classification of the user into the one or more of the patient subgroups, wherein the action comprises one or more of the following: (i) displaying a notification to the user, via a display of the device, of at least one risk associated with the one or more patient subgroups, (ii) presenting to the user, via a display of the device, of educational content associated with the at least one risk, (iii) presenting to the user, via a display of the device, of contact information for a healthcare provider specialized for the one or more eye health conditions, and (iv) establishing a communicative connection between the device and a remote server.

2. The method of claim 1, wherein the behavioral data regarding the user's use of the device comprises a measurement of time spent viewing the device or a measurement of time spent in an outdoors space.

3. The method of claim 1, wherein the remote server is the computing system or telecommunications system of an eye health care provider.

4. The method of claim 3, wherein the performing of an action in response to the classification of the user into the one or more of the patient subgroups comprises presenting the user with an interface for scheduling an in-person appointment with the eye health care provider.

5. The method of claim 1, wherein the remote server is a purchasing system or a distribution system.

6. The method of claim 1, wherein the remote server is the computing system of an educational provider.

7. A method comprising:
capturing, by a device, behavioral data regarding (a) a user's use of the device, or (b) the user's interaction with the user's environment;
capturing, by the device, one or more of
(i) input data comprising one or more of: (a) user survey data, (b) user biometric data, (c) data entered by the user through a screen or peripheral of the device, and (d) device sensor data and
(ii) medical data for the user relating to one or more eye health conditions, wherein the first remote system contains records from at least one eye health care provider;
obtaining, from a remote system, historical data related to one or more of: (a) medical history of the user, (b) medical data relating to a plurality of patients, (c) clinical trial data, and (d) purchase data;
providing the historical data, the behavioral data, and one or more of the input data and the medical data to a machine learning model, the machine learning model having been trained to generate a health score associated with the user; and
displaying a notification to the user, via a display of the device, of information associated with the generated health score.

8. The method of claim 7, wherein the generated health score is any of: an eye health score, an overall health score, a behavioral health score, and a cardiovascular health score.

9. The method of claim 7, wherein the generated health score is an eye health score.

10. The method of claim 9, wherein the eye health score represents a progression of an eye condition over time.

11. The method of claim 9, wherein the eye health score represents a comparison of an eye health of the user against an eye health value representative of an aggregate patient population.

12. A method comprising:
capturing, via a device, behavioral data regarding (a) a user's use of the device, or (b) the user's interaction with the user's environment;
providing the behavioral data to a machine learning model, the machine learning model having been trained to classify the user into one or more of the patient subgroups, each patient subgroup being associated with a distinct eye health condition;
establishing, in response to the classification of the user into the one or more of the patient subgroups, a communicative connection between the device and a computing system or telecommunications system of an eye health care provider; and
providing to the user, in response to the classification of the user into the one or more of the patient subgroups, an interface with an appointment scheduling system for the eye health care provider.

13. The method of claim 12, further comprising providing, to the user, a listing of suggested eye health care providers.

14. The method of claim 12, wherein the appointment scheduling system is a system for scheduling an in-office appointment with the eye health care provider.
